(19) United States
(12) Patent Application Publication
(10) Pub. No.: US 2024/0325016 A1
(43) Pub. Date: Oct. 3, 2024

(54) DEVICE FOR RELOADING SUTURE ANCHOR ONTO A TISSUE REPAIR DEVICE

(71) Applicant: Biomet Manufacturing, LLC., Warsaw, IN (US)

(72) Inventor: Preston McCurdy, Fort Wayne, IN (US)

(21) Appl. No.: 18/615,153

(22) Filed: Mar. 25, 2024

Related U.S. Application Data
(60) Provisional application No. 63/455,862, filed on Mar. 30, 2023.

(51) Int. Cl. A61B 17/04

(52) U.S. Cl. CPC A61B 17/0401; A61B 2017/0409; A61B 2017/0414; A61B 2017/0446

(57) ABSTRACT

A system for tissue repair may include a suture anchor, a tissue repair device and an apparatus. The tissue repair device optionally including a handle, a shaft, and an end effector configured to selectively couple with the suture anchor. The apparatus configured to selectively carry the suture anchor and one or more sutures coupled to the suture anchor. The apparatus configured to pass the suture anchor from the apparatus and onto the end effector.

### CLAIM OF PRIORITY

This application claims the benefit of U.S. Provisional Patent Application Ser. No. 63/455,862, filed on Mar. 30, 2023, the benefit of priority of which is claimed hereby, and which is incorporated by reference herein in its entirety.

### FIELD OF THE DISCLOSURE

The present disclosure relates to devices used for bone or tissue repair surgery including a device for reloading a suture anchor onto a tissue repair device.

### BACKGROUND OF THE DISCLOSURE

In the human body, tissue can require repair. Such tissue includes bone, muscles, tendons, ligaments and cartilage. Forceful twisting, trauma or rotation of the knee, shoulder (or other joint) can tear or otherwise damage tissue. A surgical repair of the tissue may be required. Such repair can include suturing and anchoring to bone. Various assemblies have been developed for facilitating suturing and are effective for their intended purposes. Nevertheless, improvements to tissue repair devices for facilitating suturing are still desirable.

### SUMMARY

Tissue repair devices (commonly called inserters) are used for suture anchor deployment into tissue (including bone or soft tissue). Although inserters have been developed that can deploy multiple suture anchors into tissue, these inserters can be bulkier and/or more complex to operate than inserters configured to deploy only a single suture anchor. Thus, single suture anchor inserters have typically been preferred by many surgeons. However, single suture anchor inserters are more expensive as they utilize more material because the inserter must be disposed of after deployment of only a single suture anchor. These single use devices cannot be reused. Most surgical tissue repairs are complex and require the deployment of multiple suture anchors to anchor sutures and repair tissue in multiple locations.

The present inventor recognizes an apparatus such as a package or card that can facilitate deployment of multiple suture anchors using an inserter typically configured for only single suture anchor deployment. This allows what would otherwise be a single use inserter to be reused multiple times during the surgery thereby saving cost and reducing material waste. As discussed above, the single anchor inserter is typically less complex for the surgeon to operate.

The apparatus can carry a suture anchor for reloading onto the inserter and can carry one or more sutures coupled to the suture anchor. The apparatus can be configured to precisely position the suture anchor for coupling to the inserter. Additionally, the apparatus is configured to guide the inserter when coupling with the suture anchor. This positioning and guiding avoids inaccurate loading of the suture anchor onto the inserter that could otherwise occur if free-hand loading were attempted. The apparatus can be configured to retain and organize the suture(s) coupled to the anchor such that the suture(s) do not become tangled during loading of the suture anchor onto the inserter. The apparatus can include a cover to facilitate sterilized carrying of the suture anchor and sutures within the apparatus. The cover can be detachable or removable from one or more sides of the apparatus to allow for removal of the suture anchor from the apparatus (e.g., to create one or more of an inlet and/or an outlet for a track or passage of the apparatus). The design and shape of the apparatus, including the track and/or other features such as an anchor holder, can correspond to different suture anchors and/or inserter styles, shapes and sizes.

### BRIEF DESCRIPTION OF THE DRAWINGS

FIG. 1 illustrates a rotator cuff repair being performed on a shoulder joint of a patient in accordance with one example of the present application.

FIG. 2 shows a tissue repair device configured to deploy a soft anchor in accordance with one example of the present application.

FIG. 2A is an enlarged view of a distal portion of the tissue repair device of FIG. 2 additionally showing the soft anchor loaded thereon.

FIGS. 3A-3C show various sides of an apparatus configured to reload the soft anchor onto the tissue repair device of FIGS. 2 and 2A in accordance with one example of the present application.

FIG. 3D is a plan view of the apparatus of FIGS. 3A-3C from a bottom side additionally showing part of the soft anchor positioned in a track thereof in accordance with one example of the present application.

FIG. 4 is a cross-sectional view of the apparatus of FIGS. 3A-3D showing an interior of the apparatus, FIG. 4 additionally illustrates the tissue repair device, the soft anchor and an anchor holder in accordance with one example of the present application.

FIGS. 5A and 5B illustrate a process of loading the soft anchor onto the tissue repair device using the apparatus in accordance with one example of the present application.

FIG. 6 illustrates the anchor holder in accordance with one example of the present application.

FIG. 7 illustrates another example of the anchor holder according to the present application.

FIG. 8 illustrates yet another example of the anchor holder co-molded or otherwise connected with the track according to the present application.

Corresponding reference characters indicate corresponding parts throughout the several views. Inserters in the drawings are not necessarily drawn to scale. The configurations shown in the drawings are merely examples, and should not be construed as limiting the scope of the invention in any manner.

### DETAILED DESCRIPTION

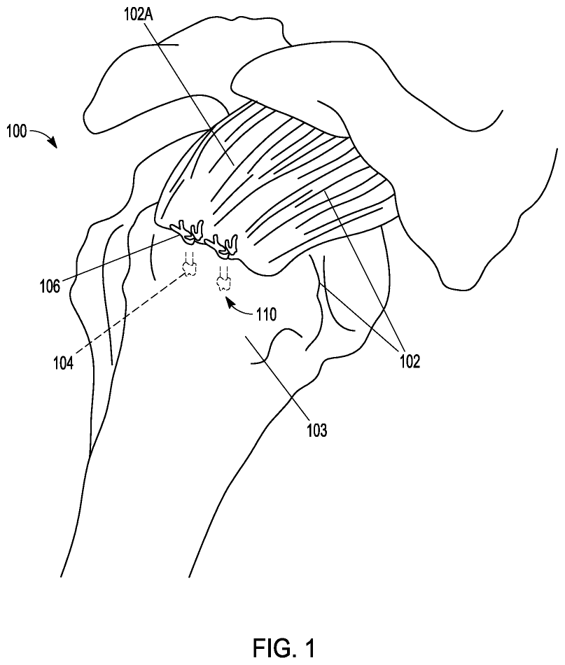

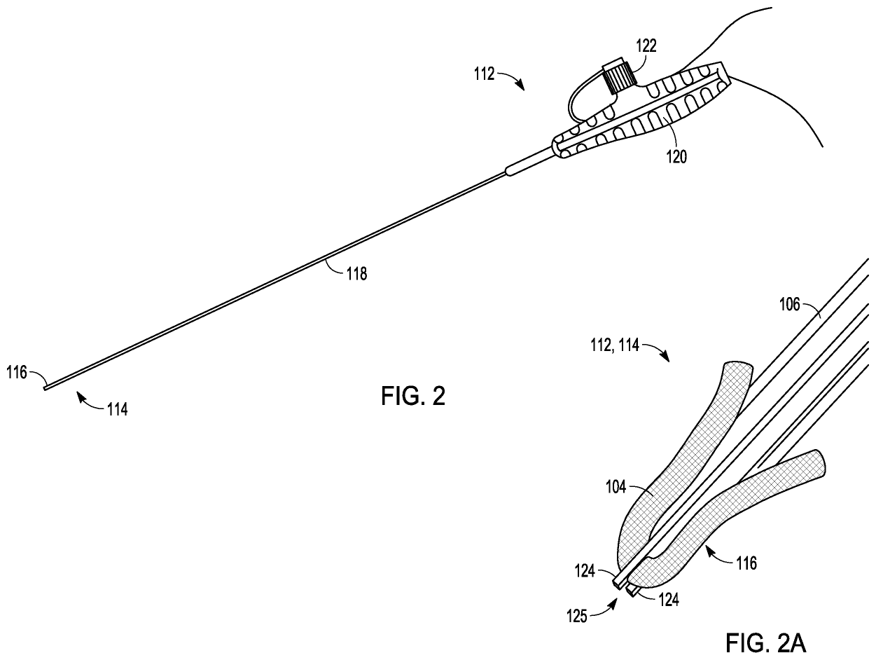

To repair tissue (including both soft tissue and bone) in the human body a surgeon will typically deploy two or more suture anchors into the tissue. Suture anchors and suture can be used to reattach muscle to bone, for example. FIG. 1 illustrates a tissue repair to a damaged shoulder joint 100 of a patient. The tissue 102 can be a damaged rotator cuff 102A, for example. According to one example, a plurality of suture anchors 104 (obscured by a humerus 103 but illustrated subsequently such as in FIG. 2A, for example) can be placed in the humerus 103 below or adjacent the rotator cuff 102A. Sutures 106 can be coupled to each of the suture anchors 104. The sutures 106 can be tied down or to one another, coupled to the rotator cuff 102A or coupled to one or more additional suture anchors or apparatuses such as a mesh (not shown). The suture anchors 104 and sutures 106 can be used to stretch the rotator cuff 102A back into a desired position and attach the rotator cuff 102A back to the humerus 103.

The surgeon can use a tissue repair device (sometimes called an inserter, deployment device or simply device herein) to aid in deploying the suture anchor(s). The tissue repair device may have or can be used in conjunction with other devices such as a device that has a needle to pierce the tissue or bone adjacent the tear or the tissue. This leaves one or more apertures 110. The tissue repair device can then be configured to deploy the suture anchors 104 into the apertures 110 as shown in FIG. 1.

The suture anchors disclosed herein can be hard or soft anchors or can be combinations thereof. The soft suture anchor(s) are referred to as “soft” herein as they are formed of material(s) that are flexible and/or deformable, such as a suture sleeve or other suture material. These soft suture anchors can be constructed as tubes that are collapsible once tension is applied to the suture 106. Use of soft suture anchors made of deformable material can be preferable to using a hard-plastic anchor, made from a relatively hard material, such as polyether ether ketone (PEEK). For example, in cases where the suture pulls through the tissue, the suture anchor can be dislodged within the joint or surgical space. In such instances, chances of damage is reduced with the soft suture anchor as compared with the hard suture anchor. Soft anchors are known and include the JuggerKnot® Soft Anchor manufactured and sold by Zimmer Biomet of Warsaw, IN. Soft suture anchor such as the suture anchor 104 shown can be an elongated member having first and second ends. The first and second ends can be blunt and substantially perpendicular to a longitudinal axis of the suture anchor 104. The suture anchor 104 can be made of resorbable or non-resorbable materials, including braided suture, sponges and sponge-like materials in solid form, perforated materials, woven/braided from biocompatible materials or fibers, such as, for example, polymer, polyester, polyethylene, cotton, silk, or other natural or synthetic materials, including sponges and sponge-like materials. The suture anchor 104 can also be an elongated tubular or solid member or a two-dimensional member with or without internal bores. The suture anchor 104 can have any properties that allow the suture anchor 104 to change shape. The suture anchor 104 can be, for example, collapsible, compliant, flexible, foldable, squashable, squeezable, deformable, limp, flaccid, elastic, low-modulus, soft, spongy, perforated or any other flexible member which can change shape. In some aspects, the suture anchor 104 can be coated with biological or biocompatible coatings, and it can also be soaked in platelets and other biologics, which can be easily absorbed by the suture anchor 104.

Tissue repair devices for deploying soft anchors connected with the suture loop, such as those of application Ser. No. 15/482,106 (now U.S. Pat. No. 10,499,902) and application Ser. No. 16/251,342 (now U.S. Pat. No. 11,116,495) are known. The contents of both of these applications are incorporated herein by reference in their entirety. The present application provides further examples of an apparatus that can be used with tissue repair devices including single anchor deploying devices utilized for deploying suture anchors.

FIG. 2 shows an example of a tissue repair device 112 having a distal portion 114 with an end effector 116, a shaft 118 and a handle 120. The handle 120 can include a suture capture 122. FIG. 2A shows an enlargement of the distal portion 114 showing the end effector 116 coupled to one of the suture anchors 104. In particular, the suture anchor 104 and sutures 106 coupled thereto are carried for deployment into tissue between prongs 124 of the end effector 116. The prongs 124 can form sides of a recess, eyelet or other capture feature 125 for receiving the suture anchor 104. The tissue repair device 112 is configured for deployment of a single suture anchor, the suture anchor 104, and can then be reloaded with another suture anchor 104 using the apparatus as discussed subsequently.

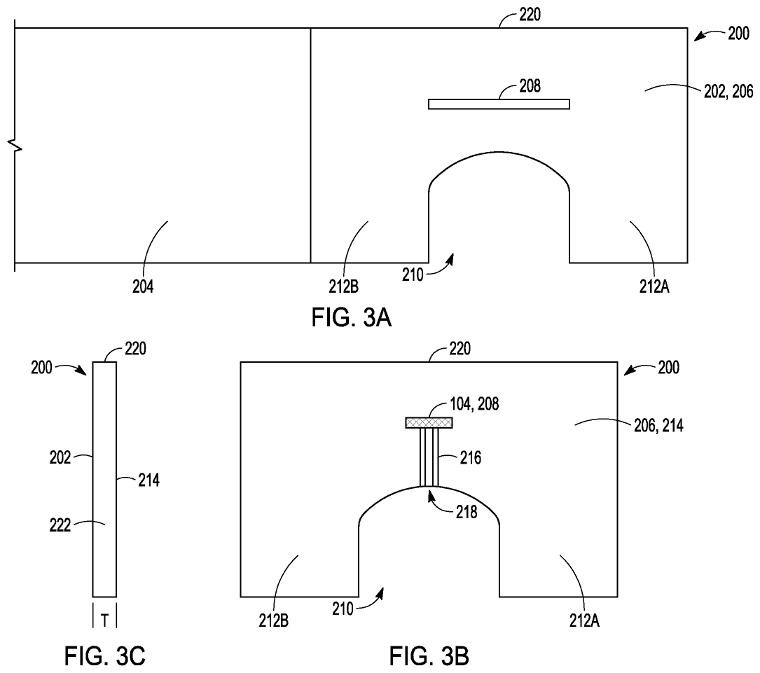

FIG. 3A is a plan view of an apparatus 200 from a first side 202. In FIG. 3A, a cover 204 (only a portion is shown) has been detached or removed from the first side 202 and from an opposing side. The apparatus 200 in addition to the cover 204 includes a body 206, an aperture 208 and a recess 210.

The first side 202 can be one of two major sides/surfaces of the apparatus 200. The first side 202 can be generally flat or can be curved or otherwise shaped as desired. The cover 204 can be constructed from a relatively thin foldable or otherwise bendable or manipulatable material such as plastic, plastic composite (e.g., paper coated in plastic, etc.). The cover 204 can be glued or otherwise attached to cover one or more sides of the body 206 including the first side 202 and/or an opposing side, for example. The body 206 can be constructed of rigid or semi-rigid material such as plastic, plastic composite (e.g., cardboard coated in plastic, paper coated in plastic, etc.) or other material(s) suitable for a surgical environment. This construct for the cover 204 and the body 206 allows the apparatus 200 to be readily sterilized for use. The cover 204 can allow sides including the first side 202 and the opposing side and internal features of the body 206 to be sealed (by cover 204) to maintain sterilization. The apparatus 200 can be constructed as a package or card as further described herein and can be configured to carry a suture anchor (e.g., the suture anchor 104 (FIG. 2A) or another suture anchor).

With the cover 204 partially removed as shown in FIG. 3A, the body 206 can have an arch shape due to the recess 210. Thus, the body 206 can include a leg 212A and a leg 212B to each side of the recess 210. The recess 210 can extend entirely through the body 206 and can extend toward a center of the body 206. The aperture 208 such as a slot can be centrally located on the first side 202 generally positioned above and adjacent an end of the recess 210. The aperture 208 can extend into an interior of the body 206. In some cases, the aperture 208 can extend through the body 206 from the first side 202 to the opposing side. The aperture 208 can be configured to receive a suture anchor holder that is insertable into the body 206 as discussed subsequently.

FIGS. 3B-3D show the apparatus 200 from different sides and with the cover 204 (FIG. 3A) entirely removed from the apparatus 200. FIG. 3B shows the opposing side 214 of the apparatus 200 from the first side 202 (FIG. 3A). FIG. 3B shows the body 206, the recess 210 and the legs 212A, 212B as previously discussed. Additionally, FIG. 3B shows a track 216 extending along and/or having an outlet 217 to the opposing side 214.

The track 216 can communicate at an opening 218 with the recess 210. This opening 218 can comprise an inlet to the track 216. The track 216 can extend generally in a straight path toward a second side 220 of the body 206 that opposes the recess 210 and the legs 212A, 212B. The second side 220 can comprise a minor side of the body 206 extending between the first side 202 (FIG. 3A) and the opposing side 214. The second side 220 can have an extent/area much smaller than that of the first side 202 (FIG. 3A) or the opposing side 214. This is due to the relatively smaller thickness dimension T (FIGS. 3C and 3D) of the apparatus 200.

The track 216 can be formed in/by the body 206 and can traverse at least a portion of the body 206. According to some examples, the track 216 can be formed of a different material from the body 206 such as metal, metal alloy, composite or another suitable material. In some cases, the track 216 can extend entirely across the body 206 from the recess 210 to the second side 220. The track 216 can communicate with the aperture 208 (only a portion shown in FIG. 3B) as the aperture 208 can intersect the track 216 and can extend through the opposing side 214.

FIG. 3C shows a third side 222 of the apparatus 200 that extends between the first side 202 and the opposing side 214. Like the second side 220 of FIG. 3B, the third side 22 can be a minor side of the body 206 extent/area much smaller than that of the first side 202 or the opposing side 214. This is due to the relatively smaller thickness dimension T of the apparatus 200.

FIG. 3D shows a fourth side 224 (also called a bottom side herein) of the apparatus 200. This fourth side 224 is shaped by the recess 210 and the legs 212A, 212B as previously discussed. The fourth side 224 extends between the first side 202 and the opposing side 214. FIG. 3D additionally shows the track 216 with the opening 218 on the fourth side 224 and the outlet 217 on the opposing side 214. The track 216 can lead to or can intersect with a portion of the suture anchor 104 that is carried by the body 206. The track 216 can terminate at or adjacent (after) intersection with the portion of the suture anchor 104. Additionally, the track 216 can terminate at a ramp or other feature adjacent the outlet 217 that facilitates the end effector 116 exiting the body 206 through the outlet 217 on the opposing side 214.

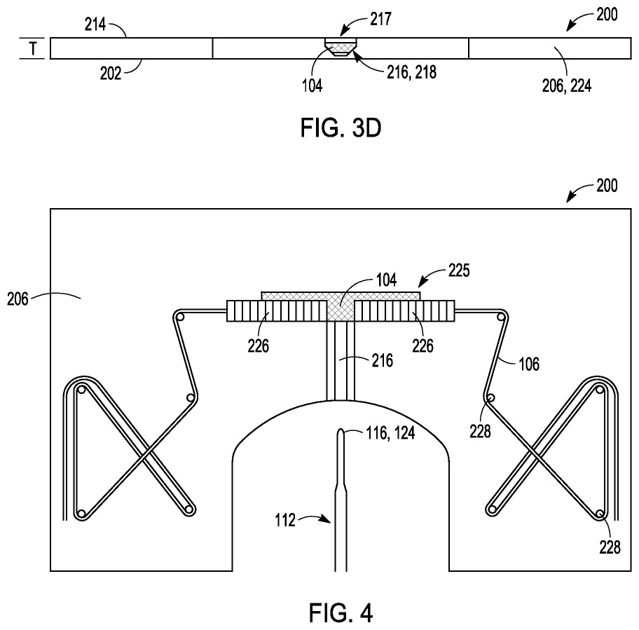

FIG. 4 shows the apparatus 200 with a surface that forms the opposing side 214 removed so as to show further components of the apparatus 200 internal to the body 206. These internal features/components include a recess 225, an anchor holder 226 and one or more suture posts or wheels 228. The suture anchor 104 and sutures 106 can be housed within the body 206 so as to be carried by the apparatus 200.

The recess 225 can be sized and shaped to receive the suture anchor 104 therein as shown in FIG. 4. The recess 225 can communicate with the track 216, for example. The anchor holder 226 (shown in cross-section) can be insertable into the body 206 and partially into the recess 225 via the aperture 208 (FIGS. 3A and 3B). However, according to other examples the anchor holder 226 can be permanently housed within the body 206 and/or can be a feature of the body 206. In further examples, an anchor holder may not be utilized in favor of other internal retention features within the body 206.

The anchor holder 226 can engage the suture anchor 104. The anchor holder 226 can compress the suture anchor 104. This can occur by bending or otherwise deforming the suture anchor 104 to assume an arcuate, V, U, S or other shape. Compression of the suture anchor 104 by the anchor holder 226 can keep the sutures 106 taut on the one or more suture posts or wheels 228. The anchor holder 226 can position the suture anchor 104 at a desired location within the body 206 such as within the recess 225 with a central portion of the suture anchor 104 being positioned within the track 216 (see e.g., FIGS. 3B, 3D and 4).

The one or more suture posts or wheels 228 can be configured to retain the suture 106 such as by partial wrapping, full wrapping or the like. The one or more suture posts or wheels 228 can be configured (e.g., can rotate, can be positioned, can engage, etc.) to allow the suture 106 to unwind in a controlled manner that keeps tension on the suture anchor 104, for example. The one or more suture posts or wheels 228 are optional and may not be utilized according to some examples.

FIG. 4 additionally shows the suture anchor 104 and sutures 106 and the end effector 116 of the tissue repair device 112 beginning the process of being reloaded with the suture anchor 104. The track 216 can be configured to receive at least a portion of the end effector 116 and the shaft 118 of the tissue repair device 112. In some cases, the track 216 can be configured (e.g., positioned, shaped and sized) to guide the end effector 116 to the suture anchor 104 (partially shown) when the end effector 116 is in a first orientation relative to the apparatus. This first orientation can align the prongs 124 (only one is shown in FIG. 4) so that the suture anchor 104 can be received in the recess, eyelet or other capture feature 125 (FIG. 2A) between the prongs 124 of the end effector 116.

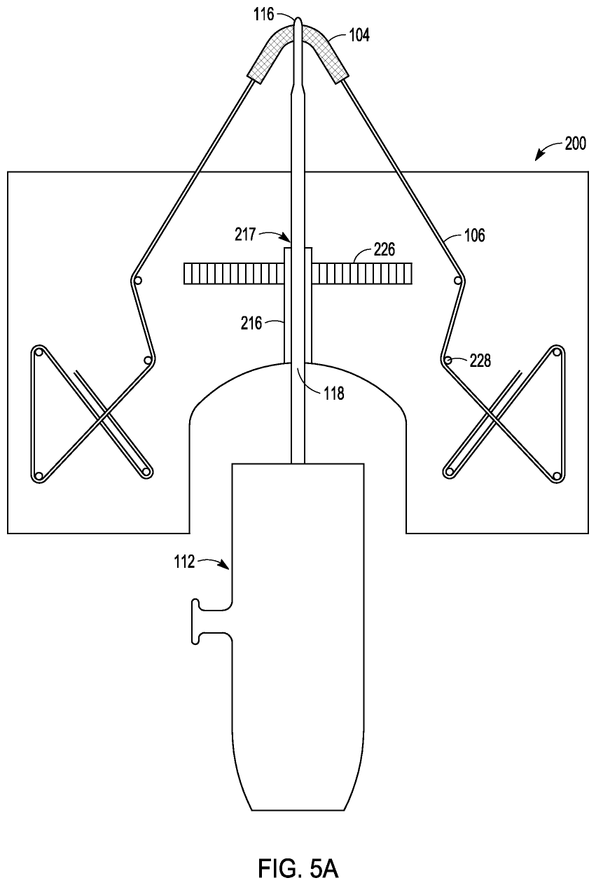

FIGS. 5A and 5B illustrate further the process of the suture anchor 104 and sutures 106 reloading with the tissue repair device 112 and the suture anchor 104 and sutures 106 being removed from the apparatus 200.

FIG. 5A shows the shaft 118 of the tissue repair device 112 inserted further through the apparatus 200 along the track 216 then in FIG. 4. The suture anchor 104 is coupled to the end effector 116. A distal portion of the shaft 118 and end effector 116 have exited the apparatus 200 via the outlet 217 to the track 216. The sutures 106 have begun the process of unwinding on the one or more suture posts or wheels 228. The anchor holder 226 remains in the body 206.

FIG. 5B shows the suture anchor 104 and sutures 106 coupled to the tissue repair device 112. The sutures 106 can be coupled to the suture capture 122 of the handle 120. A feature such as a removable cap 300 can be used to keep the sutures 106 coupled to the suture capture 122. The suture anchor 104 and sutures 106 are ready for deployment into tissue via the tissue repair device 112 as previously discussed herein.

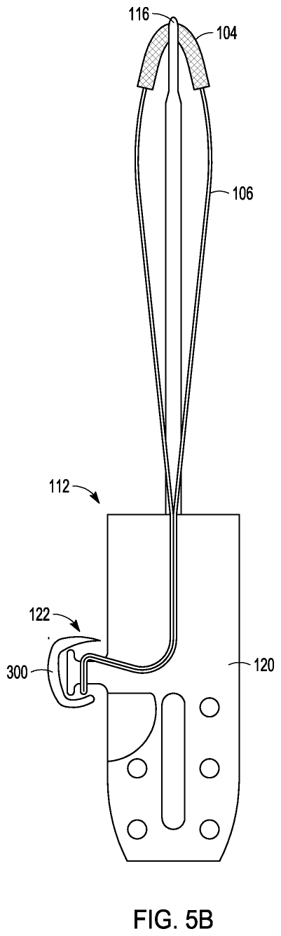

FIG. 6 shows a first example of the anchor holder 226. The anchor holder 226 can include an engagement feature 400, a stop 402 and a handle 404. The handle 404 in coupled to the engagement feature 400 and the stop 402. The engagement feature 400 can be configured to engage the suture anchor and can be configured to compress the suture anchor as discussed previously. The shape of the engagement feature 400 can be different from the arcuate or crescent shape shown in FIG. 6. In some cases, the shape of the engagement feature 400 can be dictated by the shape of the suture anchor, the track or combination thereof. The stop 402 can be configured to provide the engagement feature 400 with a desired known depth of insertion into the body of the apparatus as previously illustrated and discussed. The stop 402 can also be configured as a locking feature to lock the anchor holder 226 within a desired position within the body. The position and/or shape of the stop 402 can differ from different sizes and shapes of suture anchor. The handle 404 provides for grasping of the anchor holder 226 for insertion and/or removal of the anchor holder 226 from the apparatus by the surgeon or other operating room personnel.

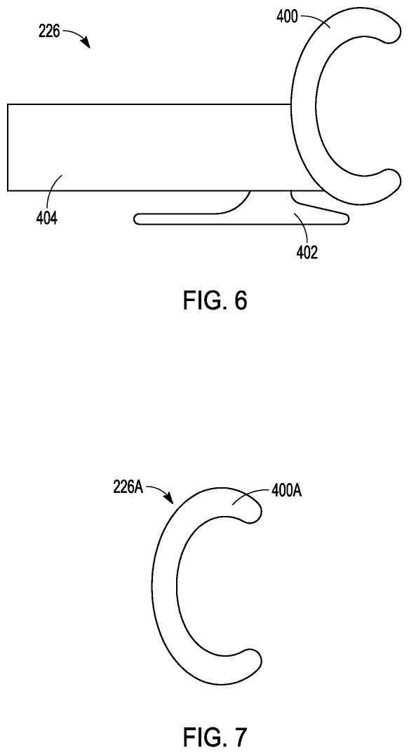

FIG. 7 shows another example of an anchor holder 226A. This anchor holder 226A only includes an engagement feature 400A and is not configured for insertion and/or removal from the apparatus such as via the aperture. Rather, the anchor holder 226A can be permanently or temporarily housed within the body 206 so as to be a feature of the body 206. The engagement feature 400A can be configured in the manner of the engagement feature 400 so as to engage and compress the suture anchor as discussed previously.

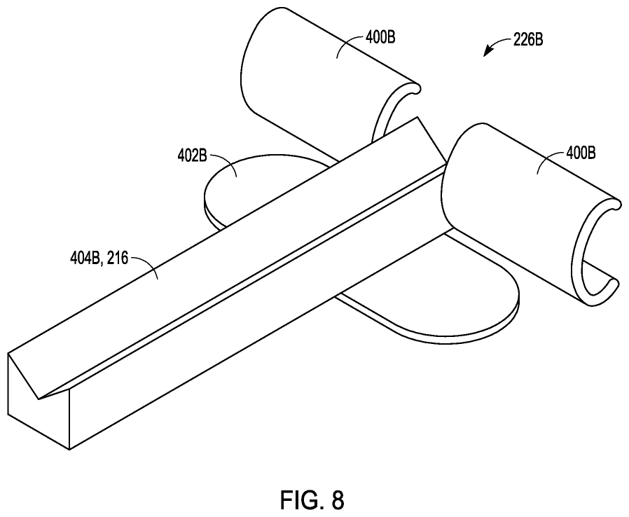

FIG. 8 shows yet another example of an anchor holder 226B. The anchor holder 226B can be co-molded together so as to form both an engagement feature 400B and the track 216 discussed previously. Optionally, the track 216 could serve as a handle 404B for personnel. A stop 402B can be utilized to anchor the anchor holder 226B to the remainder of the apparatus, for example.

The surgeon can choose to position the two or more anchors in a suitable pattern that is matched to address the injury and complete a repair. In some examples, the surgeon can position one anchor above the other. In other examples, the surgeon can position the anchors side by side. Anchors can be or may not be connected to together with strands of suture cinched or otherwise coupled or may not be connected together. Other examples contemplate the use of the suture anchors and sutures with a device such as an orthopedic mesh.

As discussed previously, the suture anchors 104, etc. discussed herein can be “soft” (i.e., they can be made from a relatively soft material such as fabric or suture), and thus they can bend, flex and/or deform under the force of a suture or inserter(s) on the repair device. In some examples, the suture anchors 104, etc. can be nominally shaped as cylinders, or tubes having a circular or elongated cross-section, but can be configured to deform such as by collapsing during deployment.

Many of the FIGURES herein illustrate components of the repair device including the anchors in a highly schematic manner. This is done to better illustrate interaction of various components and the tissue repair device. However, it is recognized that the suture anchors 104, etc. can have other shapes and can be deformable as discussed above. Similarly, other components can have shapes different from those illustrated herein.

According to other examples contemplated herein not all anchors may not be soft and can be made from non-deformable material such as a hard plastic, etc. Thus, according to some examples the anchor(s) can have a cross-section or other geometry that is invariant. In these examples, the anchor(s) can have a hollow interior formed by a tubular shape of the anchor(s). The suture anchors 104, etc. can be differently sized/shaped relative to the examples provided.

It will be understood that the foregoing are merely examples, and that other deployment schemes and device configurations can also be used. Furthermore, combinations of the various foregoing examples can be used together as desired according to further contemplated examples. It will be understood that one of ordinary skill in the art can modify the foregoing devices to achieve a desirable deployment of the first anchor and the second anchor.

While this invention has been described as having example designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### Claims Related Examples/Aspects/Techniques.

To further illustrate the devices, systems and related methods disclosed herein, a non-limiting list of examples (referred to as aspects and techniques) is provided below. Each of the following non-limiting examples can stand on its own, or can be combined in any permutation or combination with any one or more of the other examples.

In some aspects, the techniques described herein relate to a system for tissue repair optionally including: a suture anchor; a tissue repair device including a handle, a shaft, and an end effector configured to selectively couple with the suture anchor; and an apparatus configured to selectively carry the suture anchor and one or more sutures coupled to the suture anchor, the apparatus configured to pass the suture anchor from the apparatus and onto the end effector.

In some aspects, the system, further optionally including an anchor holder configured to engage and compress the suture anchor.

In some aspects, the system, wherein optionally the anchor holder is selectively insertable into the apparatus to position the suture anchor at a desired location.

In some aspects, the system, wherein optionally the apparatus includes at least one of a plurality of posts or one or wheels to retain the one or more sutures.

In some aspects, the system, wherein optionally the apparatus is configured as a package having cover that is selectively removable from at least a portion of the apparatus.

In some aspects, the system, wherein optionally the apparatus includes a track configured to receive at least a portion of the end effector and the shaft of the tissue repair device, and wherein the track is configured to guide the end effector to the suture anchor when the end effector is in a first orientation relative to the apparatus.

In some aspects, the system, wherein optionally the suture anchor is a soft anchor formed of deformable material.

In some aspects, the techniques described herein relate to an apparatus for selectively carrying a suture anchor, optionally including: a body; a recess within the body receiving the suture anchor; a track formed in the body and traversing at least a portion of the body, wherein the track intersects with the recess; and a holder configured to engage and selectively position the suture anchor relative to the track.

In some aspects, the apparatus, wherein optionally the apparatus is configured to retain one or more sutures coupled to the suture anchor.

In some aspects, the apparatus, wherein optionally the apparatus includes at least one of a plurality of posts or one or wheels to retain the one or more sutures.

In some aspects, the apparatus, wherein optionally the apparatus is configured as a package constructed of a plastic or plastic composite.

In some aspects, the apparatus, wherein optionally the apparatus includes a cover that is selectively removable from at least a portion of a side of the body.

In some aspects, the apparatus, wherein optionally the holder is configured to position at least a portion of the suture anchor within the track, and wherein the track is configured to receive at least a portion of an end effector of a tissue repair device.

In some aspects, the apparatus, wherein optionally the holder is one of a feature internal to the body or insertable into the body through an aperture in a side thereof.

In some aspects, the apparatus, wherein optionally the body is shaped as an arch with a recess to accommodate insertion of a tissue repair device along the track.

In some aspects, the techniques described herein relate to a method of loading a suture anchor onto a tissue repair device, the method optionally including: carrying the suture anchor within an apparatus; inserting an end effector of the tissue repair device into the apparatus; and passing the suture anchor from the apparatus to the end effector with the end effector inserted into the apparatus.

In some aspects, the method, further optionally including positioning the suture anchor within a track of the apparatus.

In some aspects, the method, wherein optionally the inserting the end effector of the tissue repair device is guided by the track.

In some aspects, the method, further optionally including retaining one or more sutures coupled to the suture anchor with the apparatus.

In some aspects, the method, further optionally including removing a cover from the apparatus to allow for removal of the suture anchor from the apparatus.

What is claimed is:

1. A system for tissue repair comprising:
a suture anchor;
a tissue repair device including a handle, a shaft, and an end effector configured to selectively couple with the suture anchor; and
an apparatus configured to selectively carry the suture anchor and one or more sutures coupled to the suture anchor, the apparatus configured to pass the suture anchor from the apparatus and onto the end effector.

2. The system of claim 1, further comprising an anchor holder configured to engage and compress the suture anchor.

3. The system of claim 2, wherein the anchor holder is selectively insertable into the apparatus to position the suture anchor at a desired location.

4. The system of claim 1, wherein the apparatus includes at least one of a plurality of posts or one or wheels to retain the one or more sutures.

5. The system of claim 1, wherein the apparatus is configured as a package having cover that is selectively removable from at least a portion of the apparatus.

6. The system of claim 1, wherein the apparatus includes a track configured to receive at least a portion of the end effector and the shaft of the tissue repair device, and wherein the track is configured to guide the end effector to the suture anchor when the end effector is in a first orientation relative to the apparatus.

7. The system of claim 1, wherein the suture anchor is a soft anchor formed of deformable material.

8. An apparatus for selectively carrying a suture anchor, comprising:
a body;
a recess within the body receiving the suture anchor;
a track formed in the body and traversing at least a portion of the body, wherein the track intersects with the recess; and
a holder configured to engage and selectively position the suture anchor relative to the track.

9. The apparatus of claim 8, wherein the apparatus is configured to retain one or more sutures coupled to the suture anchor.

10. The apparatus of claim 9, wherein the apparatus includes at least one of a plurality of posts or one or wheels to retain the one or more sutures.

11. The apparatus of claim 8, wherein the apparatus is configured as a package constructed of a plastic or plastic composite.

12. The apparatus of claim 8, wherein the apparatus includes a cover that is selectively removable from at least a portion of a side of the body.

13. The apparatus of claim 8, wherein the holder is configured to position at least a portion of the suture anchor within the track, and wherein the track is configured to receive at least a portion of an end effector of a tissue repair device.

14. The apparatus of claim 13, wherein the holder is one of a feature internal to the body or insertable into the body through an aperture in a side thereof.

15. The apparatus of claim 8, wherein the body is shaped as an arch with a recess to accommodate insertion of a tissue repair device along the track.

16. A method of loading a suture anchor onto a tissue repair device, the method comprising:
carrying the suture anchor within an apparatus;
inserting an end effector of the tissue repair device into the apparatus; and
passing the suture anchor from the apparatus to the end effector with the end effector inserted into the apparatus.

17. The method of claim 16, further comprising positioning the suture anchor within a track of the apparatus.

18. The method of claim 17, wherein the inserting the end effector of the tissue repair device is guided by the track.

19. The method of claim 16, further comprising retaining one or more sutures coupled to the suture anchor with the apparatus.

20. The method of claim 16, further comprising removing a cover from the apparatus to allow for removal of the suture anchor from the apparatus.
